(19) United States
(12) Patent Application Publication
(10) Pub. No.: US 2020/0230164 A1
(43) Pub. Date: Jul. 23, 2020

(54) ACTIVATION OF iNKT CELLS

(71) Applicants: INSERM (INSTITUT NATIONAL DE LA SANTE ET DE LA RECHERCHE MEDICALE), Paris (FR); CENTRE NATIONAL DE LA RECHERCHE SCIENTIFIQUE (CNRS), Paris (FR); UNIVERSITE DE LILLE 2, UNIVERSITE DU DROIT ET DE LA SANTE, Lille (FR); THE LEIDEN UNIVERSITY MEDICAL CENTER, Leiden (NL); INSTITUT PASTEUR DE LILLE, Lille (FR)

(72) Inventors: Francois TROTTEIN, Lille (FR); Christelle FAVEEUW, Lille (FR); Elodie MACHO FERNANDEZ, Lille (FR); Luis Javier CRUZ RICONDO, Leiden (NL)

(21) Appl. No.: 16/751,638

(22) Filed: Jan. 24, 2020

Related U.S. Application Data
(63) Continuation of application No. 14/766,913, filed on Aug. 10, 2015.
(60) Provisional application No. 61/766,789, filed on Feb. 20, 2013.

(30) Foreign Application Priority Data
Feb. 20, 2013 (EP) 13155949.4

(51) Int. Cl. A61K 31/7032; C08G 63/08; A61K 47/68; A61K 47/69; A61K 31/7028

(52) U.S. Cl. CPC A61K 31/7032; C08G 63/08; A61K 31/7028; A61K 47/6937; A61K 47/6843; A61K 47/6849

(57) ABSTRACT

The present invention relates to particulate entity, such as a nanoparticle or conjugate, for use in particular as adjuvant in vaccine or immunotherapy. More specifically, the invention relates to a particulate entity comprising: iv. an iNKT cell agonist such as α Gal Car compound, and, v. one or more antigenic determinant(s) such as a tumour antigen(s) or pathogen-derived antigen(s), vi. a targeting agent that targets in vivo said iNKT cell agonist to dendritic cells, such as human BDCA3-dendritic cells.

### FIELD OF THE INVENTION

The present invention relates to particulate entity, such as a nanoparticle or conjugate, for use in particular as adjuvant in vaccine or immunotherapy. More specifically, the invention relates to a particulate entity comprising:
i. an iNKT cell agonist such as the prototypical iNKT cell ligand α-galactosylceramide (α-GalCer),
ii. optionally, one or more antigenic determinant(s), such as a tumour antigen(s) or pathogen-derived antigen(s), and,
iii. a targeting agent that targets in vivo said iNKT cell agonist(s) and, optionally, said one or more antigenic determinant(s), to dendritic cells, such as human BDCA3+ dendritic cells.

### BACKGROUND OF THE INVENTION

Immunotherapy against cancer remains a promising approach to control tumor growth and hold great promises for induction of antitumor immunity.

Invariant Natural Killer T (iNKT) cells represent a population of non-conventional T lymphocytes possessing “innate-like” functions and playing positive and negative roles in numerous pathologies, including cancer, infections, inflammation and autoimmune diseases.¹⁻³ Invariant NKT cells express NK lineage receptors and a semi-invariant TCRα chain that pairs with a limited number of Vβ chains. This cell population recognizes, through their TCR, self and exogenous lipid Ag presented by the CD1d molecule expressed by Ag presenting cells (APC).^{4.5} In response to the prototypical iNKT cell activator α-galactosylceramide (α-GalCer), iNKT cells rapidly produce a wide array of immunostimulatory cytokines, including IFN-γ and IL-4, and up-regulate several costimulatory molecules.² These events contribute to the reciprocal maturation of APC, for instance the release of IL-12 by dendritic cells (DCs), and to the downstream activation of NK cells, γδ T cells and B and T lymphocytes, with important outcomes on immune responses.^{1-3,6} Through this activation cascade, α-GalCer and α-GalCer analogues are viewed as potent adjuvants for vaccine or therapy in cancer.^{1,2,7}

Conventional DCs (termed DCs) are believed to be the main players in the initiation of the iNKT cell response and in downstream activation of by-stander cells in response to α-GalCer.⁸⁻¹² Dendritic cells are heterogeneous and can be classified into different subtypes according to their phenotype, tissue distribution and functions.^{13,14} Dendritic cells in the spleen, an important site of immune responses to blood-borne Ag,¹⁵ are mainly composed of CD8α-DCs, encompassing CD4+ and CD4-subsets, and of CD8α+ DCs, expressing or not the CD103 and CD207 molecules.^{16,17} CD8α+ DCs, and most particularly the CD207+ fraction, are specialized for cross-presentation, whereas CD8α-DCs are more efficient at presenting Ag on MHC class II.^{13,16,18,19} The nature of DC subsets that participate in the initiation of the iNKT cell response remains largely unknown. Previous studies have suggested that after systemic administration of α-GalCer, CD207+ CD8α+ DCs are dispensable for the initial activation of iNKT cells whereas they play a critical role, through IL-12p70 release, in IFN-γ production by NK cells.^{18,20}

Previous works in mice have shown that a single administration of α-GalCer induces iNKT cell anergy, defined by their inability to proliferate and produce IFN-γ upon secondary stimulation.^{10,11} This property strongly precludes the clinical use of α-GalCer in humans.^{21,22} Several reports suggested that the presentation of α-GalCer by inappropriate CD1d-bearing APCs, including B lymphocytes, might lead to iNKT cell anergy.^{10,11,23} In contrast, α-GalCer presentation by DCs appears to avoid iNKT cell anergy,^{10,11} although this has recently been called into question.²³ Thus, the role of DCs in iNKT cell anergy is still an open question.

An objective of the present invention is to provide improved immune-based therapies and vaccines, particularly in cancer patients. More specifically, the present invention is based on a controlled delivery of iNKT cell agonist, such α-GalCer, optionally together with one or more tumour antigen(s) into certain APCs for efficient iNKT activation and, at later time points, for efficient iNKT cell-mediated adaptive immune responses.

### SUMMARY OF THE INVENTION

The invention relates to a particulate entity comprising:
i. an iNKT cell agonist,
ii. optionally, one or more antigenic determinant(s), and,
iii. a targeting agent that targets in vivo said iNKT cell agonist and, optionally, said one or more antigenic determinant(s), to human dendritic cells.

In one specific embodiment, said iNKT cell agonist is α-GalCer molecule or its functional derivatives. In another specific embodiment, said antigenic determinant(s), is (are) a tumour or pathogen-derived antigen. In another specific embodiment, said targeting agent is targeting said iNKT cell agonist to human BDCA3+ dendritic cells. In a more specific embodiment, said particulate entity does not comprise CD1d molecule.

Accordingly, in one preferred embodiment, the invention relates to a particulate entity comprising:
i. an α-GalCer compound consisting of α-galactosylceramide or its functional derivatives capable of activating invariant natural killer T (iNKT) cells, and,
ii. optionally, one or more antigenic determinants, such as tumour antigen(s) or pathogen-derived antigen(s),
iii. a targeting agent that targets in vivo said α-GalCer and optionally, said one or more antigenic determinant(s), to human BDCA3+ dendritic cells,

In one embodiment, said particulate entity is a nanoparticle having a size between 10 to 2000 nm diameter. Typically, said nanoparticle comprises a core containing polymers and a coating, wherein said targeting agent is covalently linked to the surface of the coating. In a specific related embodiment, said core of the nanoparticle, comprises poly(lactic acid), poly(glycolic acid), or their co-polymers.

In another embodiment, said particulate entity is a conjugate consisting of said iNKT cell agonist, for example α-GalCer compound, covalently linked to the targeting agent, optionally via a linker.

α-galactosylceramide may consist of (2S,3S,4R)-1-O-(alpha-D-galactosyl)-N-hexacosanoyl-2-amino-1,3,4-octadecanetriol or its functional derivatives that activates iNKT cells.

In embodiments that may be combined with the preceding embodiments, said targeting agent comprises a binding molecule that specifically binds to a cell surface marker of human dendritic cells, including BDCA-3+ dendritic cells. BDCA3+ dendritic cells are Lin-(CD3, C14, CD16, CD19, CD20, CD56), HLA-DR+, BDCA3+ (also known as CD141), Clec9A+, XCR-1+, TLR3+, CD11c+. Accordingly, in one specific embodiment, said targeting agent is a binding molecule to a cell surface marker specific of BDCA-3+ dendritic cells. For example, said marker specific of BDCA3+ dendritic cells is selected from the group consisting of XCR-1 and CLEC9A (also known as DNGR-1).

In other embodiments that may be combined with the preceding embodiments, a binding molecule for use as targeting agent is an antibody that binds specifically to at least one of the cell surface markers specific of human BDCA-3+ dendritic cells.

In a specific embodiment that may be combined with the preceding embodiments, said particulate entity does not comprise CD1d molecule.

In another embodiment, the particulate entity further comprises an antigenic determinant. Said antigenic determinant may be specific for an infectious agent, a pathogen, a fungal cell, a bacterial cell, a viral particle or a tumor cell.

This invention also relates to a pharmaceutical composition, comprising a particulate entity as described above, and one or more physiologically acceptable excipients. The composition may further comprise the iNKT cell agonist with an antigenic determinant, and/or other immune stimulants, including without limitation agonist of the Toll-like receptor and/or the NOD-like receptor families.

The particulate entity according to the invention or the pharmaceutical composition are particularly useful either
i. as an adjuvant in a vaccine composition;
ii. in preventing or treating cancer or infection disorders; or,
iii. in preventing or treating autoimmune and inflammatory disorders such as asthma.

More specifically, the particulate entity or composition of the invention may be used in methods for preventing or treating tumour development or infectious diseases.

### DETAILED DESCRIPTION OF THE INVENTION

The inventors indeed investigated the possibility that active in vivo α-GalCer and antigens targeting to dendritic cells, by means of antibody (Ab)-armed nanoparticles (NPs), might improve iNKT cell-dependent immune responses. Using PLGA-based nanoparticles carrying on their surface targeting agent to CD8α+ murine DCs, they show for the first time that the in vivo delivery of α-GalCer compound and antigen into CD8a+ DCs not only enhance the early activation of iNKT cells and, at later time points, iNKT cell-mediated adaptive immune responses (B and T cell responses) but also allows iNKT cells to respond to further re-stimulation, paving the way to new strategies for cancer therapy and vaccination.

Thus, in one aspect, the invention provides a particulate entity comprising:
i. an invariant Natural Killer T (iNKT) cell agonist, and,
ii. optionally, one or more antigenic determinants, and,
iii. a targeting agent that targets in vivo said iNKT cell agonist and optionally said one or more antigenic determinant(s) to dendritic cells.
   iNKT Cell Agonist

As used herein, the term “iNKT cell agonist” has its general meaning in the art and refers to any derivative or analogue derived from a lipid, that is typically presented in a CD1d context by antigen presentating cells (APCs) and that can activate iNKT cells, i.e. promote, in a specific manner, cytokine production by iNKT cells. Typically the iNKT cell agonist is a α-galactosylceramide compound.

As used herein, the term “α-galactosylceramide compound” or “α-GalCer compound” has its general meaning in the art and refers to any functional derivative or analogue derived from a glycosphingolipid that contains a galactose carbohydrate attached by an α-linkage to a ceramide lipid that has an acyl and sphingosine chains of variable lengths (Van Kaer L. α-Galactosylceramide therapy for autoimmune diseases: Prospects and obstacles. Nat. Rev. Immunol. 2005; 5: 31-42).

A functional derivative retains the capacity to activate iNKT cells.

Various publications have described α-GalCer compounds and their synthesis. An exemplary, but by no means exhaustive, list of such references includes Morita, et al., J. Med. Chem., 25 38:2176 (1995); Sakai, at al., J. Me d. Chem., 38:1836 (1995); Morita, et al., Bioorg. Med. Chem. Lett., 5:699 (1995); Takakawa, et al., Tetrahedron, 54:3150 (1998); Sakai, at al., Org. Lett., 1:359 (1998); Figueroa-Perez, et al., Carbohydr. Res., 328:95 (2000); Plettenburg, at al., J. Org. Chem., 67:4559 (2002); Yang, at al., Angew. Chem., 116:3906 (2004); Yang, at al., Angew. Chem. Int. Ed., 43:3818 (2004); and, Yu, etal., Proc. Natl. Acad. Sci. USA, 102(9):3383-3388 (2005).

Examples of patents and patent applications describing instances of α-GalCer compounds include U.S. Pat. Nos. 5,936,076; 6,531,453 5,S53,737 , 8,022,043, US Patent Application 2003030611, US Patent Application 20030157135, US Patent Application 20040242499, US Patent Application 20040127429, US Patent Application 20100104590, European Patent EP0609437 and International patent application WO2006026389.

A typical α-GalCer compound is KRN7000 ((2S 3S, 4R)-1-0-(alfaD-galactopyranosyl)-N-hexacosanoyl-2-amino-1,3,4-octadecanetriol)) (KRN7000, a novel immunomodulator, and its antitumor activities. Kobayashi E, Motoki K, Uchida T, Fukushima H, Koezuka Y. Oncol Res. 1995;7(10-11):529-34.).

Other Examples Include:

(2S,3R)-1-(α-D-galactopyranosyloxy)-2-tetracosanoylamino-3-octadecanol,

(2S,3R)-2-docosanoylamina-1-(α-D-galactopyranosyloxy)-3-octadecanol,

(2S,3R)-1-(α-D-galactopyranosyloxy)-2-icosanoylamino-3-octadecanol,

(2S,3R)-1-(α-D-galactopyranosyloxy)-2-octadecanoylamino-3-octadecanol,

(2S,3R)-1-(α-D-galactopyranosyloxy)-2-tetradecanoylamino-3-octadecanol,

(2S,3R)-2-decanoylamino-1-(α-D-40galactopyranosyloxy)-3-octadecanol,

(2S,3R)-1-(α-D-galactopyranosyloxy)-2-tetracosanoylamino-3-tetradecanol,

(2S,3R)-1-(α-D-galactopyranosyloxy)-2-tetradecanoylamino-3-hexadecanol,

(2R,3S)-1-(α-D-galactopyranosyloxy)-2-tetradecanoylamino-3-hexadecanol,

(2S,3S)-1-(α-D-galactopyranosyloxy)-2-tetradecanoylamino-3-hexadecanol,

(2S,3R)-1-(α-D-galactopyranosyloxy)-2[(R)-2-hydroxytetracosanoylamino]-3-octadecanol,

(2S,3R,4E)-1-(α-D-galactopyranosyloxy)-2-octadecanoylamino-4-octadecen-3-ol,

(2S,3R,4E)-1-(α-D-galactopyranosyloxy)-2-tetradecanoylamino-4-octadecen-3-ol,

(2S,3S,4R)-1-(α-D-galactopyranosyloxy)-2-tetracosanoylamino-3,4-octadecanediol,

(2S,3S,4R)-1-(α-D-galactopyranosyloxy)-2-tetracosanoylamino-3,4-heptadecanediol,

(2S,3S,4R)-1-(α-D-galactopyranosyloxy)-2-tetracosanoylamino-3,4-pentadecanediol,

(2S,3S,4R)-1-(α-D-galactopyranosyloxy)-2-tetracosanoylamino-3,4-undecanediol,

(2S,3S,4R)-1-(α-D-galactopyranosyloxy)-2-hexacosanoylamino-3,4-heptadecanediol,

(2S,3S,4R)-1-(α-D-galactopyranosyloxy)-2-[(R)-2-hydroxytetracosanoylamino]-3,4-octadecanediol,

(2S,3S,4R)-1-(α-D-galactopyranosyloxy)-2-[(R)-2-hydroxytetracosanoylamino]-3,4-heptadecanediol,

(2S,3S,4R)-1-(α-D-galactopyranosyloxy)-2-[(R)-2-hydroxytetracosanoylamino]-3,4-pentadecanediol,

(2S,3S,4R)-1-(α-D-galactopyranosyloxy)-2-[(R)-2-hydroxytetracosanoylamino]-3,4-undecanediol,

(2S,3S,4R)-1-(α-D-galactopyranosyloxy)-2-[(R)-2-hydroxyhexacosanoylamino]-3,4-octadecanediol,

(2S,3S,4R)-1-(α-D-galactopyranosyloxy)-2-[(R)-2-hydroxyhexacosanoylamino]-3,4-nonadecanediol,

(2S,3S,4R)-1-(α-D-galactopyranosyloxy)-2-[(R)-2-hydroxyhexacosanoylamina]-3,4-icosanediol,

(2S,3S,4R)-1-(α-D-galactopyranosyloxy)-2-[(S)-2-hydroxytetracosanoylamino]-3,4-heptadecanediol,

(2S,3S,4R)-1-(α-D-galactopyranosyloxy)-2-[(R)-2-hydroxytetracosanoylamino]-3,4-hexadecanediol,

(2S,3S,4R)-1-(α-D-galactopyranosyloxy)-2-[(S)-2-hydroxytetracosanoylamino]-16-methyl-3,4-heptadecanediol,

(2S,3S,4R)-1-(α-D-galactopyranosyloxy)-16-methyl-2-tetracosanoylamino-3,4-heptadecanediol,

(2S,3S,4R)-1-(α-D-galactopyranosyloxy)-2-[(R)-2-hydroxytricosanoylamino]-16-methyl-3,4-heptadecanediol,

(2S,3S,4R)-1-(α-D-galactopyranosyloxy)-2-[(R)-2-hydroxypentacosanoylamino]-16-methyl-3,4-octadecanediol,

(2S,3R)-1-(α-D -galactopyranosyloxy)-2-oleoylamino-3-octadecanol,

(2S,3S,4R)-1-(α-D-galactopyranosyloxy)-2-hexacosanoylamino-3,4-octadecanediol;

(2S,3S,4R)-1-(α-D-galactopyranosyloxy)-2-octacosanoylamino-3,4-heptadecanediol

(2R,3R)-1-(α-D-galactopyranosyloxy)-2-tetradecanoylamino-3-hexadecanol

(2S,3R,4S,5R)-2-((2S,3S,4R)-2-(4-hexyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-2-(4-heptyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-2-(4-hexadecyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-3,4-dihydroxy-2-(4-tricosyl-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-tetracosyl-1H ,1,2,3-triazol-1-yl)octadecyloxy)-6-(hydroxymethyl)-tetrahydro-2H-pyrane-3,4,5-triol;

(2S,3R,4S,5R)-2-((2S,3S,4R)-3,4-dihydroxy-2-(4-pentacosyl-1H-1,2,3-triazol-1-yl)octadccyloxy)-6-(hydroxymethyl)-tetrahydro-28-pyrans-3,4,5-triol;

(2S,3R,4S,5R)-2-((2S,3S,4R)-3,4-dihydroxy-2-(4-(6-phenylhexyl)-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-(7-phenylheptyl)-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-(8-phenyloctyl)-1H-1,2,3-triazol-1-yl)octadecyloxy)-6-(hydroxymethyl)-tetrahydro-28-pyrans-3,4,5-triol;

11-amino-N-((2S,3S,4R)-3,4-dihydroxy-1-((2S,3R,4S,5R)-3,4,5-trihydroxy-6-(hydroxymethyl)-tetrahydro-28-pyran-2-yloxy)octadecan-2-yl)undecanamide;

12-amino-N-((2S,3S,4R)-3,4-dihydroxy-1-((2S,3R,4S,5R)-3,4,5-trihydroxy-6-(hydroxymethyl)-tetrahydro-2H-pyran-2-oxy)octadecan-2-yl)dodecanamide;

N-((2S,3S,4R)-3,4-dihydroxy-1-((2S,3R,4S,5R)-3,4,5-trihydroxy-6-(hydroxymethyl)-tetrahydro-2Hpyran-2-yloxy)octadecan-2-yl)-11-hydroxyundecanamide;

N-((2S,3S,4R)-3,4-dihydroxy-1-((2S,3R,4S,5R)-3,4,5-trihydroxy-6-(hydroxymethyl)-tetrahydro-2Hpyran-2-yloxy)octadecan-2-yl)-12-hydroxydodecanamide;

8-(diheptylamino)-N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)octanamide;

N-((2S,3R,4S,5R)-3,4-dihydroxy-1((2S,3R,4S,5R)-3,4,5-trihydroxy-6-(hydroxymethyl)-tetrahydro-2Hpyran-2-yloxy)octadecan-2-yl)-11-(dipentylamino)undecanamide;

11-(diheptylamino)-N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)undecanamide;

N-((2S,3S,4R)-3,4-dihydroxy-1-((2S,3R,4S,5R)-3,4,5-trihydroxy-6-(hydroxymethyl)-tetranydro-2Hpyran-2-yloxy)octadecan-2-yl)-11-mercaptoundecanamide;

N-((2S,3S,4R)-3,4-dihydroxy-1-((2S,3R,4S,5R)-3,4,5-dihydroxy-6-(hydroxymethyl)-tetrahydro-2Hpyran-2-yloxy)octadecan-2-yl)-12-mercaptododecanamide,

In some embodiments α-GalCer compounds are pegylated. As used herein, the term “pegylated” refers to the conjugation of a compound moiety (i.e. α-GalCer compound) with conjugate moiety(ies) containing at least one polyalkylene unit. In particular, the term pegylated refers to the conjugation of the compound moiety (i.e. α-GalCer compound) with a conjugate moiety having at least one polyethylene glycol unit.

Derivatives of α-galactosylceramide also include functional derivatives of α-galactosylceramide which have been modified for chemical coupling (conjugation) to another molecule.

The phrase “activate iNKT cells” or “induce iNKT immune response” have similar meanings and refer for instance to the observed induction of cytokine production, such as IFN-γ in iNKT cells by α-GalCer compound. Analysis of cytokine (e.g. IFN-γ) production by iNKT cells can be performed by intracellular flow cytometry using PBS-57-loaded CD1d tetramer and TCRβ antibody

In one specific embodiment, the particulate entity according to the invention comprises (2S,3S,4R)-1-O-(alpha-D-galactosyl)-N-hexacosanoyl-2-amino-1,3,4-octadecanetriol or its functional derivative.

The Targeting Agent

It is an important finding of the invention that the efficient delivery of iNKT cell agonist, such as α-GalCer compound, optionally with one or more antigenic determinant(s), to specific dendritic cells, and in particular to murine CD8α+ or the human equivalent BDCA3+ dendritic cells, allows to enhance the early activation of iNKT cells while allowing iNKT cells to respond to further re-stimulations.

Accordingly, the particulate entity of the invention comprises a targeting agent that targets in vivo said iNKT cell agonist, optionally together with one or more antigenic determinant(s), such as tumor antigens or pathogen-derived antigens, to dendritic cells, such as human BDCA3+ dendritic cells or related cells in other mammalian species with similar phenotype, such as CD8α+ dendritic cells in murine species.

In one embodiment, said targeting agent is a molecule that specifically binds to a cell surface marker of human dendritic cells. In specific embodiment, said targeting agent specifically hinds to a cell surface marker of human BDCA3+ dendritic cells.

In one embodiment, a “cell surface marker” of human BDCA3+ dendritic cells refers to a protein or a biomolecule of human BDCA3+ dendritic cells, that is expressed on the external surface of BDCA3+ cells. More specifically, it may correspond to an antigenic determinant of BDCA3+ cells that is expressed on the surface of BDCA3+ dendritic cells and can be recognized specifically by antibodies. Preferably, the targeting agent hinds to a cell surface marker that is specific of BDCA3+ cells, i.e. that is not expressed on other dendritic cells (or at a lower level). In one specific embodiment, said targeting agent is binding to a cell surface marker specific of BCDA3+ cells, wherein said cell surface marker is not expressed on CLEC9A negative cells.

In one embodiment, a molecule that specifically binds to a cell surface marker of human BDCA3+ dendritic cells is a molecule that binds to the extracellular domain of said cell surface marker of human BDCA3+ dendritic cells, with a K_{D} of 100 μM or less, 10 μM or less, 1 μM or less, 100 nM or less, or 10 nM or less. The term K_{D} as used herein, is intended to refer to the dissociation constant, which is obtained from the ratio of K_{d} to Kₐ (i.e. K_{d}/Kₐ) and is expressed as a molar concentration (M). K_{D} values can be determined using methods well established in the art. A method for determining the K_{D} of a molecule, such as a protein or an antibody, is by using surface plasmon resonance, or using a biosensor system such as a Biacore® system.

BDCA3+ dendritic cells are Lin− (CD3, C14, CD16, CD19, CD20, CD56), HLA-DR+, BDCA3+ (also known as CD141), Clec9A+, XCR-1+, TLR3+, CD11c+. Accordingly, in one specific embodiment, said targeting agent is a binding molecule to a cell surface marker specific of BDCA-3+ dendritic cells selected from the group consisting of CLEC9A (such as human CLEC9A of SEQ ID NO:1) or XCR-1 (such as human XCR-1 of SEQ ID NO:2). Accordingly, in one embodiment, the particulate entity comprises, as a targeting agent, a molecule that binds specifically to CLEC9A and/or to XCR-1, typically, to the extracellular domain of CLEC9A or to the extracellular domain of XCR-1.

Any molecule known to have binding specificity towards a cell surface marker of human dendritic cells, preferably towards human BDCA3+ specific cell surface marker, can be used for preparing the particulate entity of the invention. Antibodies are particularly appropriate since antibodies with desired binding specificity may be routinely generated, for example by screening antibody libraries against the desired target. Screening methods may include for example, phage display technologies or other related technologies known in the Art. Such antibodies may also be easily grafted to nanoparticles or directly conjugated to the iNKT cell agonist, such as α-GalCer compound, using conventional chemical coupling technologies.

Therefore, in a preferred embodiment, the particulate entity of the invention comprises, as a targeting agent, an antibody or its antigen-binding fragments, that binds specifically to a cell surface protein of human BDCA3+ dendritic cells, such as anti-XCR-1 or anti-CLEC9A antibodies, for example with a K_{D} of at least 100 μM or less, 10 μM or less, 1 μM or less, 100 nM or less, or 10 nM or less.

As used herein, the term antibody includes full-length antibodies and any antigen-binding fragment or single chains thereof. A “full-length” antibody is a glycoprotein comprising at least two heavy (H) and two light (L) chains inter-connected by disulphide bonds. Each heavy chain is comprised of heavy chain variable region (abbreviated as VH) and a heavy chain constant region which comprises three domains, CHE CH2, and CH3. Each light chain is comprised of a light chain variable region (abbreviated as VL) and a light chain constant region comprising one domain (CL). The VH and VL domains are further subdivided into 3 regions of hypervariability, termed complementary determining regions (CDRs), interspersed with regions that are more conserved, termed framework regions (FR). Each VH and VL is therefore composed of three CDRs and four FRs arranged from amino-terminus to carboxy-terminus in the following order: FR1, CDR1, FR2, CDR2, FR3, CDR3, FR4.

Commercially available antibodies or their derivatives may also be used in the particulate entities of the invention. Anti-CLEC9A antibodies are available for example from Miltenyi Biotec (Germany).

Nanoparticle Carrying iNKT Cell Agonist, Optionally the Antigenic Determinant(s) and the Targeting Agent

For efficient targeting of iNKT cell agonist (for example α-GalCer compound) and optionally the antigenic determinant(s), by the targeting agent, said iNKT cell agonist and optionally said antigenic determinant(s) must be coupled to the targeting agent either by indirect or direct coupling, thereby forming a particulate entity. An example of indirect coupling is the encapsulation of the iNKT cell agonist (for example α-GalCer compound), optionally the antigenic determinant(s), in a nanoparticle further carrying the targeting agent for proper in vivo delivery of said iNKT cell agonist, and optionally said antigenic determinant(s), to suitable dendritic cells. In such embodiment, the iNKT cell agonist (for example α-GalCer compound), optionally, the one or more antigenic determinant(s), and the targeting agent are physically associated to the same particulate entity, i.e. the nanoparticle.

Ideally, the nanoparticle may have the following features:
it is biocompatible,
it can physically couple the iNKT cell agonist, optionally the antigenic determinant(s), and the targeting agent via covalent or non-covalent linkage.

“Physical coupling” may result from either covalent binding of the targeting agent and/or iNKT cell agonist (for example α-GalCer compound) and optionally, the antigenic determinant(s) to a constituent of the nanoparticle or via non-covalent, such as electrostatic or ionic interactions.

Any nanoparticles which have been described in the art for in vivo delivery of active principles in human may be used. Such nanoparticles include for example liposomes and micelles, nanosphere or nanoparticles, nanotubes, nanocrystals, hydrogels, carbon-based nanoparticles and the like (see for example Peer et al., 2007, Nature nanotechnology, vol. 2, pp751-760).

Examples of suitable nanoparticles are also described for example in Cruz et al J Control Release 2010, 144(2):118-26.

Preferably the nanoparticle according to the invention has a mean diameter between 1 to 2000 nm diameter, for example between 10 to 500 nm or between 10 to 200 nm.

As used herein, the size of a nanoparticle may correspond to the mean value±SD of ten readings from dynamic light scattering measurements as described in Cruz et al, 2011, Cruz et al., 2010^{30,31}.

The nanoparticles of the invention may comprise an inorganic core, such as, but not limited to, semiconductor, metal (e.g. gold, silver, copper, titanium, nickel, platinum, palladium and alloys), metal oxide nanoparticles (e.g. Cr₂O₃, CO₃O₄, NiO, MnO, CoFe₂O₄, and MnFeO₄).

In other embodiments, the nanoparticles comprises at least a core with one or more polymers, or their copolymer, such as, e.g., one or more of dextran, carboxymethyl dextran, chitosan, trimetylchitosan, polyvinylalcohol (PVA), polyanhydrides, polyacylates, polymethacrylates, polyacylamides, cellulose, hydromellose, starch, dendrimers, polyamino acids, polyethyleneglycols, polyethyleneglycol-co-propyleneglycol, aliphatic polyesters, including poly(lactic acid (PLA), poly(glycolic acid), and their copolymers including poly(lactic-co-glycolylic)acid (PLGA), or poly(ε-caprolactone).

In general the surface of the nanoparticles may also be functionalised or coated to produce a desirable physical characteristic such as solubility, biocompatibility, and for facilitating chemical linkages with other biomolecules, such as iNKT cell agonist, the antigenic determinant(s), or the targeting agent.

For example, the surface of the nanoparticles can be functionalized by incorporating one or more chemical linkers such as, without limitation: carboxyl groups, amine groups, carboxyl/amine, hydroxyl groups, polymers such as silane, dextran or PEG or their derivatives.

In a specific embodiment, nanoparticle has a core that comprises polymers selected from the group consisting of: poly(lactic acid), poly(glycolic acid), or mixtures thereof. In another specific embodiment, the nanoparticle comprise poly(lactic)poly(glycolic) acid co-polymers (PLGA).

Other suitable polymers may comprise polyamino acid selected from the group consisting of poly(g-glutamic acid), poly(a-aspartic acid), poly(e-lysine), poly(a-glutamic acid), poly(a-lysine), poly-asparagine, or derivatives thereof, and mixtures thereof.

In a specific embodiment, the nanoparticles of the invention comprise a core containing polymers and a coating, and the targeting agent is attached to the nanoparticle by covalent linkage to the surface of the coating. In a further specific embodiment, the nanoparticles comprises
(i) a core made of poly(lactic acid), poly(glycolic acid), or their copolymers, with a coating on its surface,
(ii) an efficient amount of iNKT cell agonist, for example, an α-GalCer compound,
(iii) optionally, an efficient amount of one or more antigenic determinant(s), for example, a tumor antigen or pathogen-derived antigen,
(iv) an antibody covalently attached to the coating of the nanoparticle, wherein said antibody binds specifically to BDCA3+ dendritic cells.

In one specific embodiment, said antibody comprised in the nanoparticles does not bind to CLEC9A-negative or XCR1-negative dendritic cells.

In a more specific embodiment, said antibody binds specifically to CLEC9A or XCR1 cell surface markers as expressed on BDCA3+ dendritic cells.

Other suitable nanoparticles include oxide and hybrid nanostructures such as iron oxide nanoparticle or polymer-based nanoparticle, optionally coated with organic or inorganic stabilizers, such as silane, dextran or PEG (see e.g. S. Chandra et al./Advanced Drug Delivery Rev (2011), doi:10.1016/j.adr.2011.06.003).

Methods for encapsulating or chemically coupling iNKT cell agonist, such as α-GalCer compound, and/or optionally, one or more antigenic determinant(s), such as antigens expressed by tumour cells or by pathogens, and/or the targeting agent to the nanoparticles are known in the art. For example, the nanoparticle is prepared together with α-GalCer compound and, optionally one or more antigenic determinant(s) and the α-GalCer compound and, optionally, said one or more antigenic determinant(s) are encapsulated (retained by non-covalent binding) into the nanoparticle. Alternatively, the nanoparticle is prepared and the iNKT cell agonist, such as α-GalCer compound, and optionally, said one or more antigenic determinant(s), are chemically linked to the functionalized surface of the nanoparticle, via conventional coupling techniques. Example of preparation of PLGA based nanoparticles, with encapsulated α-GalCer is described in Cruz et al, 2011 [Mol Pharm 2011, 8:520-531], and Cruz et al. 2010 [J Control Release 2010, 144:118-126].

In one specific embodiment, the nanoparticle comprises encapsulated α-GalCer at amounts comprised between 0.01 and 1000 ng per mg of nanoparticle. In a specific embodiment, 1 ng to 1000 ng of iNKT cell agonist per mg of nanoparticles is used. In a specific embodiment, the nanoparticle of the invention further comprises an antigenic determinant as described more in detail in the next sections. Such antigenic determinant may be encapsulated or attached to the surface of the nanoparticle, similarly to the targeting agent.

### Conjugates

Alternatively, the particulate entity of the invention results from the chemical coupling of iNKT cell agonist, such as α-GalCer compound, to the targeting agent, either directly or optionally via a linker, to form a conjugate.

Such conjugate is therefore obtained by coupling (either by covalent or non-covalent coupling) of iNKT cell agonist with the targeting agent, optionally via a linker.

The covalent linkage between iNKT cell agonist and the targeting agent is typically obtained via the use of a coupling or cross-linking agent, and optionally a linker for covalent linkage of both molecules while maintaining their functionality, or allowing cleavage. A variety of coupling or cross-linking agents can be used for making the conjugates of the invention. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g. Karpovsky ef a/., 1984 J. Exp. Med. 160: 1686; Liu, M A ef a/., 1985 Proc. Natl. Acad. Sci. USA 82:8648). Other methods include those described in Paulus, 1985 Behring Ins. Mitt. No. 78,1 18-132; Brennan ef a/., 1985 Science 229:81-83), and Glennie ef a/., 1987 J. Immunol. 139: 2367-2375). Examples of linker types include, but are not limited to, hydrazones, thioethers, esters, disulfides and peptide-containing linkers. A linker can be chosen that is, for example, susceptible to cleavage by low pH within the lysosomal compartment or susceptible to cleavage by proteases.

For a reference for methods of coupling α-GalCer compound to other compounds, see for example Bioorg Med Chem Lett. 2004 14(2):495-8 and Daoudi et al, 1999, Bioconjug Chem. 10(6):1021-31.

For covalent conjugation of α-GalCer compound to the targeting agent, biotinylated α-GalCer compound may be associated with streptavidin-antibodies or avidin-antibodies (McReynolds et al, Bioconjugate Chem., 1999, 10 (6), pp 1021-1031 DOI: 10.1021/bc990050x).

In one specific embodiment, the conjugate comprises at least an antibody molecule as targeting agent that is covalently conjugated to the iNKT cell agonist, such as α-GalCer compound. Methods for preparing conjugates with antibody molecules, also referred as immunoconjugates or ADC (antibody-drug-conjugates) have been widely described in the art.

Techniques for conjugating therapeutic agents to proteins, and in particular to antibodies, are well-known in the art and described for example in Flygare et al (Chem Biol Drug Des 2013; 81: 113-121).

In one embodiment, the conjugate comprise one molecule of iNKT cell agonist (e.g. α-GalCer compound) conjugated to one molecule of targeting agent (for example anti-CLEC9A or anti-CXR1 antibody). In specific embodiments, the conjugate may comprise more than one α-GalCer compounds conjugated to more than one targeting agent.

In a specific embodiment, the conjugate of the invention comprises one or more iNKT cell agonists, for example, one or more α-GalCer compounds, which are covalently linked to one or more anti-XCR-1 or anti-CLEC9A antibody.

### Antigenic Determinant

The particulate entity of the invention may be used as an adjuvant, i.e., for potentiating an immune response against an antigenic determinant. Accordingly, the particulate entity of the invention can be administered with an antigen either as two separate pharmaceutical compositions, or as part of the same composition, or as part of the same particulate entity. If administered separately, both compositions may be administered sequentially or simultaneously. In a specific embodiment, the antigen and the particulate entity are administered simultaneously and for example, formulated in the same composition. In other embodiment, the antigen is comprised in the particulate entity, such as the nanoparticle or the conjugate.

The resulting particulate entity or compositions with an antigenic determinant may be immunogenic, meaning that it is capable of eliciting a humoral or cellular immune response, preferably both, with respect to said antigenic determinant. Preferably, the antigenic determinant is not capable, when administered alone to induce an effector immune response. Accordingly, as used herein, the term “antigenic determinant” or “antigen” refers to any agent (e.g. protein, peptide, polysaccharide, glycoprotein, glycolipid, nucleic acid, or combination thereof) that, when introduced into a host or animal or human, having an immune system is capable of specifically interacting with an antigen recognition molecule of the immune system, such as an immunoglobulin (antibody) or T cell antigen receptor (TCR). An antigen may not be itself immunogenic and the particulate entity of the invention, such as the nanoparticle or conjugate, is used as an adjuvant, i.e. enabling to augment (potentiate) the host immune response to the antigenic determinant when administered conjointly.

The antigenic determinant comprises “epitope” which consist of portion of the antigen that are recognized by B cells or T cells, or both. For example, interaction of such epitope with an antigen recognition site of an immunoglobulin (antibody) or T cell antigen receptor (TCR) leads to the induction of antigen-specific immune response.

The antigenic determinant used in the composition or with the particulate entity according to the invention may be derived from or specific of tumor cells, i.e, it is a tumor antigen. As used herein, the term “tumor antigen” includes both tumor specific antigen (TSA) and tumor associated antigen (TAA). A tumor specific antigen is known as an antigen that is expressed only by tumor cells while tumor associated antigen are expressed on tumor cells but may also be expressed on some normal cells. Tumor specific antigens and tumor associated antigens have been described in the art. Such tumor antigen can be, but is not limited to human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, GD2, GD3, GM2, TF, sTn, MAGE-1, MAGE-3, tyrosinase, gp75, Melan-A/Mart-1, gp100, HER2/neu, EBV-LMP 1 & 2, HPV-F4, 6, 7, prostatic serum antigen (PSA), alpha-fetoprotein (AFP), CO17-1A, GA733, gp72, p53, the ras oncogene product, proteinase 3, Wilm's tumor antigen-1, telomerase, HPV E7 and melanoma gangliosides, as well as any other tumor antigens now known or identified in the future.

Other antigenic determinant include without limitation, antigens of parasite or fungus (such as candida, trichophyton), bacterial cell (e.g staphylococcus, pneumoccus or streptococcus cell, Borrelia, pseudomonas, listeria), viral particle (e.g. HIV, FIBV, HPV, HSV, HVT, CMV, HTLV, hepatitis C virus, rotavirus, flavivirus, rous associated virus, or SARS virus, yellow fever virus or dengue virus), or any portion thereof.

In a specific embodiment, said antigenic determinant is a pathogen-derived antigen. As used herein, a pathogen-derived antigen refers to an antigen that is expressed by a pathogen and not expressed on mammalian cells, in particular human cells. For example, it is an antigen expressed by viral, bacterial, or fungal pathogen of mammals.

Pharmaceutical Compositions

The invention provides pharmaceutical composition, comprising the particulate entity of the invention, containing iNKT cell agonist, optionally the antigenic determinants and the targeting agent to dendritic cells, as described in the previous sections, and one or more physiologically acceptable excipients.

In one specific embodiment, the invention relates to a pharmaceutical composition, comprising a particulate entity, with at least the following three components as described in the previous sections:
iNKT cell agonist, such as α-GalCer compound,
optionally one or more antigenic determinant(s),
the targeting agent to dendritic cells, preferably specifically to BDCA3+ dendritic cells,

wherein said composition is capable of inducing an immune response against said antigen.

The compositions of the invention are especially useful for administration to an individual in need of immune stimulation (for example for treating or preventing from infectious disease, cancer and/or allergic disorders) and comprises an efficient amount of the particulate entities according to the invention, for example, of nanoparticles or conjugates as described in the previous sections.

The compositions of the invention can be formulated using one or more physiologically acceptable excipient. Suitable excipients are for example, water, saline, buffered saline, dextrose, glycerol, ethanol, sterile isotonic aqueous buffer or the like and their combinations. In addition, if desired, the formulation may also include auxiliary substances, such as wetting or emulsifying agents, pH buffering agents, immune stimulators or other adjuvants that enhance the effectiveness of the pharmaceutical composition or vaccine. Excipients as well as formulations for parenteral and nonparenteral drug delivery are set forth in Remington's Pharmaceutical Sciences 19^{th} Ed. Mack Publishing (1995).

For example, the vaccine and pharmaceutical compositions of the invention are formulated for administration by transdermal delivery, or by transmucosal delivery, including but not limited to, oral, buccal, intranasal, ophthalmic, vaginal, rectal, intracerebral, intradermal, intramuscular, intraperitoneal, intravenous, subcutaneous routes, by inhalation or by any other standard route for immunization.

In a preferred embodiment, the compositions of the invention can be formulated for parenteral delivery, i.e. by intravenous (i.v), subcutaneous (s.c.), intraperitoneal (i.p.), intramuscular (i.m), subdermal (s.d) or intradermal.

The particular dosage regimen, i.e. dose, timing and repetition will depend on the particular individual and that individual's medical history.

The invention also relates to a kit comprising one or more containers filled with one or more of the following ingredients, for the preparation of the pharmaceutical or vaccine composition:
iNKT cell agonist, for example α-GalCer compound,
the targeting agent to dendritic cells, preferably to BDCA3+ dendritic cells,
optionally, one or more antigenic determinant(s),
one or more physiologically acceptable carrier or excipient,
optionally, one or more auxiliary substance.

The kit or the compositions according to the invention may be accompanied with a notice, in the form prescribed by a governmental agency regulating the manufacture, use or sale of pharmaceuticals or biological products, and/or instructions how to prepare the vaccine or pharmaceutical composition ready to use.

In vaccine composition, the composition may further comprise other suitable adjuvants or excipients.

Methods of Use

The particulate entity and pharmaceutical compositions of the invention are useful, e.g., for protecting against and/or treating various infectious disorders or for treating or preventing from tumors or cancers.

As used herein the term “treating” means preventing, reducing, alleviating or suppressing at least one of the symptoms of a disorder, in a subject suffering from such disorder.

For example, the particulate entity and/or pharmaceutical compositions of the invention may be used to treat or prevent from, viral infections (such as influenza viruses, leukemia viruses, immunodeficiency viruses such as HIV, papilloma viruses, herpes virus, hepatitis viruses, measles virus, poxviruses, mumps virus, cytomegalovirus [CMV], Epstein-Barr virus), bacteria infections (such as staphylococcus, streptococcus, pneumococcus, Neisseria gonorrhoea, Borrelia, pseudomonas, etc.), and fungal infections (such as Candida, Aspergillus spp, trichophyton, pityrosporum, etc..)

The particulate entity and/or pharmaceutical compositions of the invention may be used to treat or prevent from tumor or cancers, including without limitation, squamous cell cancer, small-cell lung cancer, non-small cell lung cancer, gastric cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma, sarcomas, haematological cancers (leukemias), astrocytomas, and head and neck cancer.

For example, the invention relates to a method of treating a subject suffering from cancer, infectious diseases and/or inflammatory (such as asthma) and autoimmune disorders, said method comprising administering to said subject a therapeutically efficient amount of a particulate entity of the invention or a pharmaceutical compositions of the invention.

More specifically, the particulate entity and/or pharmaceutical compositions of the invention may be used to treat cancer, infectious diseases, inflammatory (such as asthma) and autoimmune diseases.

In the following, the invention will be illustrated by means of the following examples and figures.

### FIGURES LEGENDS

FIG. 1: DCs are crucial for iNKT cell primo-activation and for the prevention of iNKT cell anergy. (A) Transgenic CD11c.DTR mice were injected with PBS or DT 24 h before α-GalCer inoculation (100 ng/mouse). (B) Spleen DCs were sorted on the basis of CD 11c expression and sensitized for 2 h with α-GalCer (25 ng/ml). Mice were then injected with α-GalCer-sensitized DCs or free α-GalCer (100 ng/mouse). A and B, Mice were euthanized 3 h after α-GalCer (A and B) or DC/α-GalCer (B) inoculation (primo-activation, grey) or received a second intravenous injection of α-GalCer (100 ng/mouse) 7 days later (recall response, black). In these later groups, animals were sacrificed 3 h after α-GalCer challenge. Splenic iNKT cells were then analysed for intracellular IFN-γ production. A representative experiment out of two (A) or three (B) is shown (mean±SD) (n=4). **p<0.01, p<0.05 (unpaired Student's t test).

FIG. 2: CD8α⁺ and CD8α⁻ DCs differ in their ability to activate iNKT cells. A, B and C, Mice were intravenously injected with α-GalCer (2 μg). Two hours later, splenic CD8α⁺ and CD8α⁻ DCs were sorted on the basis of CD11c, CD11b and CD8 expression (A) and co-cultured with sorted NKT cells (B) or with the α-GalCer-responsive IL-2-producing iNKT cell hybridoma DN32.D3 as a readout of Ag presentation (C). B and C, Cytokine production was quantified by ELISA. Results represent the mean±SD of four experiments. D, CD1d expression on CD8α⁺ and CD8α⁻ DCs was assessed by flow cytometry. The staining with the isotype control was identical on both DC subsets. Shown is a representative experiment out of three. E, Recipient mice were intravenously injected with Cy5-conjugated, or unconjugated as a control, α-GalCer (20 μg) and Cy5 incorporation by CD8α⁺ DCs (grey) and CD8α⁻ DCs (black) was analyzed by flow cytometry 2 h later. Shown is a representative histogram out of two independent experiments. ***p<0.001, *p<0.05 (unpaired Student's t test).

FIG. 3: Encapsulation of α-GalCer into NP/DEC205 targets CD8⁺ DCs and efficiently activate iNKT cells in vitro. A, BM-DCs (5×10⁵ cells/well) were exposed for 2 h with or without AlexaFluor 647-labelled PLGA particles armed with anti-DEC205 (NP/DEC205) or isotype control (NP/IgG) Abs, washed and labelled with anti-CD11c and anti-DEC205 Abs. AlexaFluor 647 labelling was then evaluated by flow cytometry on DEC205⁺ and DEC205⁻ BM-DCs. Shown are representative histograms of one experiment out of two. B, Spleen MNCs (1×10⁶ cells/well) were incubated for 2 h with or without AlexaFluor 647-labelled NP/DEC205 or NP/IgG. CD8α⁻ and CD8α⁺ DC populations were then discriminated on the basis of CD11c, CD11b and CD8 expression and analyzed by flow cytometry. Shown are representative histograms (left panel) and the mean percentages±SD of AlexaFluor 647 positive DCs (right panel) of two independent experiments (n=4). Of note, no differences between the two DC subsets were observed when NP/IgG were incubated with spleen cells. C, BM-DCs (1×10⁵ cells/well) were co-cultured for 24 h with the iNKT cell hybridoma DN32.D3 (1×10⁵ cells/well) in the presence of various doses of free α-GalCer or α-GalCer vectorized into NP/DEC205 or NP/IgG. Of note, when incubated with BM-DCs alone, NP/DEC205 and NP/IgG either loaded or not with α-GalCer failed to induce DC maturation (not shown). D, BM-derived DCs from WT and CD1d^{-/-} mice were cultured with DN32.D3 in the presence of free or vectorized α-GalCer (25 ng/ml) for 24 h. C and D, Production of IL-2 was quantified by ELISA. Data represent the mean±SD of four (C) and three (D) independent experiments. B-D, **p<0.01, *p<0.05 (unpaired Student's t test).

FIG. 4: Encapsulation of α-GalCer in NP/DEC205 efficiently activates iNKT cells in vivo. Mice were intravenously injected with PBS alone or α-GalCer either in a free soluble form or encapsulated into NP/DEC205 or NP/IgG (5 ng α-GalCer/mouse). A, After 3 h, mice were bled and splenic iNKT cells (TCRβ⁺ PBS57-loaded CD1d tetramer⁺) were screened for intracellular IFN-γ production (left panel). The average percentages±SD of iNKT cells positive for IFN-γ are represented. Production of IL-4 in the sera was quantified by ELISA (right panel) (n=3-8). B, Transgenic CD11c.DTR mice were injected with PBS or DT 24 h before the inoculation of NP/DEC205/α-GalCer (5 ng/mouse). The frequency±SD of IFN-γ⁺ iNKT cells is represented (n=3). C, The frequency±SD of IFN-γ⁺ NK cells (CD3ε⁻ NK1.1⁺) and γδ T lymphocytes (CD3ε⁺ TCRγδ⁺) are shown (4 h after stimulation). The expression of CD86 (expressed as MFI) by DCs (CD11c^{hi}) is depicted. Shown is a representative experiment (mean±SD) out of two (n=4). ***p<0.001, **p<0.01, *p<0.05 (unpaired Student's t test).

FIG. 5: Targeting α-GalCer into CD8⁺ DCs prevents iNKT cell anergy in vivo. Mice were intravenously injected with PBS, free α-GalCer (100 ng/mouse) or α-GalCer encapsulated into NP/DEC205 or NP/IgG (5 ng/mouse). A, After 3 h, mice were bled and splenic iNKT cells were screened for intracellular IFN-γ production. The average percentages±SD of iNKT cells positive for IFN-γ are represented (left panel). Production of IL-4 in the sera was quantified by ELISA (right panel). B, Mice received, 7 days later, a second injection of free α-GalCer (100 ng/ml) and spleen iNKT cells were screened for intracellular IFN-γ production 3 h later. Of note, mice treated 7 days earlier with NP/IgG/α-GalCer produced IFN-γ when challenged with α-GalCer. This is consistent with the fact that, at the dose used, NP/IgG/α-GalCer failed to trigger primary iNKT cell activation (FIG. 4A). A representative experiment out of two is shown (mean±SD) (n=3) C, The percentage±SD of iNKT cells expressing PD-1 is represented (7 days after α-GalCer stimulation). A representative experiment out of two is shown (n=4). **p<0.01, *p<0.05 (unpaired Student's t test).

FIG. 6. Co-encapsulation of α-GalCer and OVA in NP/DEC205 enhances CD8⁺ T cell and Ab responses. A, Mice, previously injected with CBE-labelled OT-1 cells, were subcutaneously inoculated with α-GalCer (5 ng/mouse) and OVA (250 ng/mouse) either free or co-encapsulated in NP/IgG or NP/DEC205. Three days later, the proliferation of CFSE-labelled Vα2 TCR⁺ CD8α⁺ in popliteal lymph nodes was determined by flow cytometry (mean±SD, n=4). B, Six days after immunization, mice were transferred with CFSE-labelled SIINFEKL-primed (targets) and PKH-26-labelled unprimed (controls) splenocytes. Data represent the percentage of specific lysis±SEM (n=6-8). C and D, Mice were injected twice (at day 0 and 21) with α-GalCer (100 ng/mouse) and OVA (5 μg/mouse) either free or co-encapsulated into NP/IgG or NP/DEC205. C, Spleen cells were restimulated 2 months later for 48h with SIINFEKI, (101.1 g/ml) and IFN-γ production in the supernatant was quantified (mean±SD, n=5). D, Blood were taken at day 28 and the anti-OVA IgG titers were determined (mean±SD, n=7-9). One representative experiment out of two is shown. *** P<0.001, **P<0.01, *P<0.05.

FIG. 7. A and B, Mice, previously injected with CFSE-labelled OT-I cells (5×10⁶ cells/mouse), were subcutaneously inoculated with α-GalCer (5 ng/mouse) and OVA (250 ng/mouse) either free or co-encapsulated in NP/IgG or NP/DEC205. A, Three days later, the proliferation of CFSE-labelled Vα2 TCR⁺ CD8α⁺ in popliteal lymph nodes was determined by flow cytometry. B, Popliteal LN cells were restimulated with the MHC Class I-restricted OVA peptide SIINFEKL and IFN-γ expression by Vα2 TCR⁺ CD8α⁺ was evaluated 18 h later by intracellular FACS staining. Shown are representative histograms (A) and dot plots (B) out of two independent experiments.

FIG. 8. Co-encapsulation of α-GalCer and OVA in NP/DEC205 triggers a potent anti-tumor response. Mice were injected with α-GalCer (20 ng) and OVA (1 μg) vectorized in NP/DEC205 or NP/IgG and 7 days later, animals were inoculated i.v with OVA-expressing B16F10 cells. Mice injected with free α-GalCer (200 ng/mouse) and OVA (10 μg/mouse) were used as positive controls. The mean number±SEM of B16F10 nodules are indicated (n=5). One representative experiment out of two is shown. *P<0.05.

### EXAMPLES

Materials and methods

### Mice

Six- to 8-wk-old male wild type C57BL/6 mice were purchased from Janvier (Le Genest-St-Isle, France) and RAG2^{-/-}×OTI from Jackson laboratory (St. Germain sur l'Arbresle, France). The generation of CD1d^{-/-} and CD11c-DTR mice has been already described.^{27,28} Mice were bred in our own facility in pathogen free conditions. Animals were handled and housed in accordance with the guidelines of the Pasteur institute Animal Care and Use Committee.

Reagents and Abs

α-GalCer was synthesized as previously described (51). Vybrant ODA SE Cell Tracer Kit was purchased from Life technologies (St Aubin, France). The PKH-26 labeling kit and ovalbumins (OVA) were purchased from Sigma-Aldrich (St Quentin-Fallavier, France). Cyanine (Cy)5-conjugated α-GalCer was synthesized as described.²⁹ APC-conjugated monoclonal Abs against mouse CD5, CD11e, CD86, PE-conjugated anti-NK 1.1, anti-TCRγδ, FITC-conjugated anti-CD8α, anti-TCRβ, anti-CD3ε, PerCpCy5.5-conjugated anti-CD11b, eFluor450-conjugated anti-Vα2 TCR, PE-Cy7-conjugated anti-CD11c, anti-CD8α, anti-PD-1, biotin-conjugated anti-CD1d, AlexaFluor 700-conjugated streptavidin, and isotype controls were purchased from BD Pharmingen (Le Pont de Claix, France) or Ozyme/Biolegend (Saint-Quentin-en-Yvelines, France). PerCP-eHuor710 anti-CD205 was purchased from eBioscience (Paris, France). IFN-γ (AlexaFluor 647-conjugated) and isotype controls were all purchased from Ozyme/Biolegend. PE-conjugated PBS-57 glycolipid-loaded CD1d tetramer was from the NIAID Tetramer Facility (Emory University, Atlanta, Ga.). The anti-DEC205 (CD205) and isotype control (IgG2b) Abs used to arm NPs was from BIO-X-CELL (West Lebanon, N.H.).

Preparation and Characterization of PLGA-Based Particles

Nanoparticles coated with lipid-PEG and carrying Abs were generated using the copolymer poly(lactic-co-glycolic acid) (PLGA) as described before.^{30,31} In brief, endotoxin-free OVA (5 mg, Sigma-Aldrich) and/or α-GalCer (50 μg) were encapsulated to 100 mg of PLGA. Anti-DEC205 Ab and its isotype control were attached to the lipid-PEG layer as described previously.³⁰ The presence of Abs on the particle surface was determined by Coomassie dye protein assay (Table 1). PLGA NPs were characterized by dynamic light scattering and zeta potential (Table 1).

TABLE 1
Nanoparticles were characterized by dynamic light scattering and zeta potential measurements.
Nanoparticle size data represent the mean value ± SD of ten readings from dynamic light scattering
measurements. ^{30, 31}Zeta potential data represent the mean value ± SD of five readings.
The amount of OVA antigen encapsulated inside of NPs was determined by Coomassie dye protein
assay and is depicted as the mean ± SD of two experiments. The incorporation of KRN
into NPs was total due to its hydrophobic nature. The amount of Abs introduced into the
NPs was determined by Coomassie Plus Protein Assay Reagent (Pierce).

| | α-GalCer | OVA | Nanoparticles | Poly- | Zeta | |
|---|---|---|---|---|---|---|
| | (μg/mg NP) | (μg/mg NP) | diameter ± SD | dispersity | potential | Abs |
| Samples | (% w/w) | (% w/w) | (nm) | index ± SD | (mV) ± SD | (μg/mg NP) |
| NP/α-GalCer/ | 0.5 | — | 194.2 ± 11.6 | 0.064 ± 0.033 | −9.5 ± 1.6 | 16.2 ± 3.4 |
| IgG | | | | | | |
| NP/α-GalCer/ | 0.5 | — | 190.3 ± 10.3 | 0.073 ± 0.037 | −12 ± 1.7 | 14.8 ± 2.3 |
| DEC205 | | | | | | |
| NP/α-GalCer/ | 0.5 | 23.8 | 245.21 ± 12.9 | 0.216 ± 0.085 | −11 ± 1.9 | 37.3 ± 5.2 |
| OVA/IgG | | | | | | |
| NP/α-GalCer/ | 0.5 | 23.8 | 248.18 ± 14.6 | 0.254 ± 0.098 | −14.1 ± 1.6 | 38.9 ± 4.5 |
| OVA/DEC205 | | | | | | |

Analysis of NP Uptake by DCs and DC-iNKT Co-Cultures

To assess the capacity of DCs to bind/uptake PLGA-based NPs, bone marrow-derived DCs (BM-DCs) (5×10⁵ cells/well)³² or spleen mononuclear cells (MNCs) (1×10⁶ cells/well) were exposed with AlexaFluor647-labelled particles (10 μg/ml and 100 μg/ml, respectively) during 2 h at 37° C. After extensive washes, BM-DC (CD11c⁺ DEC205^{+/-}) and spleen CD8α⁺ and CD8α⁻ DCs were analysed by flow cytometry. BM-DCs (1×10⁵ cells/well) were co-cultured for 24 h with the iNKT cell hybridoma DN32.D3 (1×10⁵ cells/well) in the presence of grading doses of free or encapsulated α-GalCer. To study the ex vivo stimulatory capacity of CD8α⁺ and CD8α⁻ DCs, mice were intravenously injected with 2 αg of α-GalCer and 2 h later, DC subsets were sorted using a FACSAria (Becton Dickinson, Md., USA) and co-cultured (7×10⁴ cells/well) with sorted hepatic NKT cells (CD5⁺ NK1.1⁺ cells, >98% pure) or DN32.D3 (1×10⁵ cells/well) for 48 h and 24 h, respectively. Production of IFN-γ, IL-4 and IL-2 was measured in the culture supernatants by ELISA (R&D systems).

FACS Analysis

Cells were resuspended in the appropriate combination of Abs to allow identification of DC subsets (anti-CD11c, anti-CD8α, anti-CD11b), iNKT cells (anti-TCRβ, PBS-57-loaded CD1d tetramer), NK cells (anti-CD3ε, anti-NK1.1) or γδ T lymphocytes (anti-TCRγδ, anti-CD3ε). Then, anti-PD-1,-CD86,-CD1d, or isotype controls were added when needed. Expression of intracellular IFN-γ was analysed as previously described.³² To measure Cy5-α-GalCer incorporation by splenic DC subsets, mice were intravenously injected with Cy5-conjugated α-GalCer (20 μg) and 2 h later, incorporation of Cy5 by spleen DC subsets was analysed by flow cytometry.

Role of DCs in iNKT Cell Activation and Anergy In Vivo

Mice were administrated intravenously with 200 μl of PBS containing 5 ng of free (or 100 ng as a control) or encapsulated α-GalCer. CD11c-DTR mice were injected with diphtheria toxin (111), as described,²⁷ 24 h before α-GalCer administration. Spleen DCs were sorted (CD11c⁺ cells), sensitized with 25 ng/ml of α-GalCer and injected intravenously to mice. To analyze the recall response, mice received a second intravenous injection of free α-GalCer (100 ng/mouse) one week later. Animals were bled and sacrificed 3 h post-treatment. Splenic iNKT cells were analysed for intracellular IFN-γ expression and IFN-γ and IL-4 concentrations in the sera were determined by ELISA.

Analysis of the CD8⁺ T Cell and Ab Responses

Mice received 5×10⁶ CFSE-labelled, OVA-specific, CD8⁺ T cells purified from RAG2^{-/-}×OT-I mice. One day later, mice were injected into the foodpads with NPs containing both α-GalCer (5 ng/mouse) and OVA (250 ng/mouse) or with the same quantity of free α-GalCer and OVA. Three days later, the proliferation of CFSE-labelled cells in the popliteal lymph nodes (LNs) was measured by flow cytometry. Expression of IFN-γ by Vα2 TCR⁺CD8α⁺ cells was determined after in vitro restimulation of popliteal LN cells with the MHC class 1-restricted OVA peptide SIINFEKL (10 μg/ml) for 18 h. For the in vivo CTL assay, mice animals were intravenously injected with a mixture of CFSE-labelled SIINFEKL-primed splenocytes and PKH-26-labelled unprimed splenocytes (2×10⁷ cells/mouse), 6 days after immunisation with the NP. Spleens were harvested 2 days later and the numbers of CFSE- and PKH-26-labelled cells were determined by flow cytometry. The percentage of specific lysis was determined as followed: [1-(ratio unprimed/ratio primed)]×100 where the ratio is equal to number of PHK-26 labelled cells/CFSE labelled cells). To analyze humoral and memory T cell responses, mice were intravenously injected twice (at day 0 and 21) with α-GalCer (100 ng/mouse) and OVA (5 μg/mouse) either free or co-encapsulated into NPs. Blood were taken at day 28 and the anti-OVA total IgGtiters were determined by ELISA. Spleen MNCs were prepared 2 months after the second immunization and in vitro restimulated with SIINFEKL for 48 h.

B16F10 Lung Metastasis Model.

Mice were injected with 2.5×10⁵ B16F10 melanoma cells expressing OVA 7 days after inoculation of free or vectorized OVA and α-GalCer. Mice were killed on day 18 and lung metastases were counted with the aid of a microscope.

Statistics

Results are expressed as the mean±SD or SEM. The statistical significance of differences between experimental groups was calculated by an unpaired Student's t test two-tailed (GraphPad Prism 4 software, San Diego, Calif.). Results with a p value of less than 0.05 were considered significant.

Results

Dendritic Cells Efficiently Activate iNKT Cells In Vivo Without Inducing Anergy

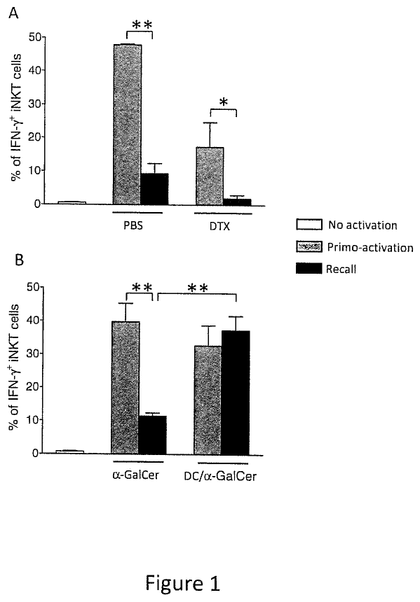

We first used transgenic CD11c.DTR mice to investigate the consequences of DC depletion on primary and secondary iNKT responses. DT treatment depleted splenic DCs (data not shown) and strongly lowered the extent of primary iNKT cell activation as exemplified by the decreased frequency of IFN-γ-positive iNKT cells (FIG. 1A) and by the reduced early release of cytokines in the sera (not shown). Whereas in DC-competent animals, iNKT cells displayed a reduced capacity to produce IFN-γ upon α-GalCer restimulation, the recall response was totally blunted when DCs were lacking at the time of primary iNKT cell stimulation (FIG. 1A). This effect was not due to a defected DC repopulation in the spleen (data not shown). Moreover, in non α-GalCer-experienced animals, this newly repopulated DC population was able to promote iNKT cell response early after α-GalCer inoculation (data not shown). Together, the lack of DCs at the time of initial iNKT cell activation led to a profound iNKT cell unresponsiveness making DCs as positive regulators of iNKT cell response upon repeated α-GalCer challenge. To confirm this finding, we investigated whether primary activation of iNKT cells by DCs could prevent iNKT cell anergy. In our experimental conditions, free α-GalCer and in vitro α-GalCer-loaded DCs triggered a similar primary activation of iNKT cells (FIG. 1B). Strikingly, whereas free α-GalCer induced the expected iNKT cell anergy, iNKT cells from mice previously inoculated with α-GalCer-loaded DCs maintained their capacity to re-activate. Of note, free α-GalCer induced enhanced PD-1 expression on iNKT cells, an inhibitory molecule that causes iNKT cell anergy,³³⁻³⁵ whereas α-GalCer-loaded DCs failed to do so (data not shown). Thus, α-GalCer presentation by DCs leads to iNKT cell activation without inducing anergy upon a subsequent challenge.

Splenic CD8α⁺ DCs Loaded In Vivo with α-GalCer Strongly Activate iNKT Cells

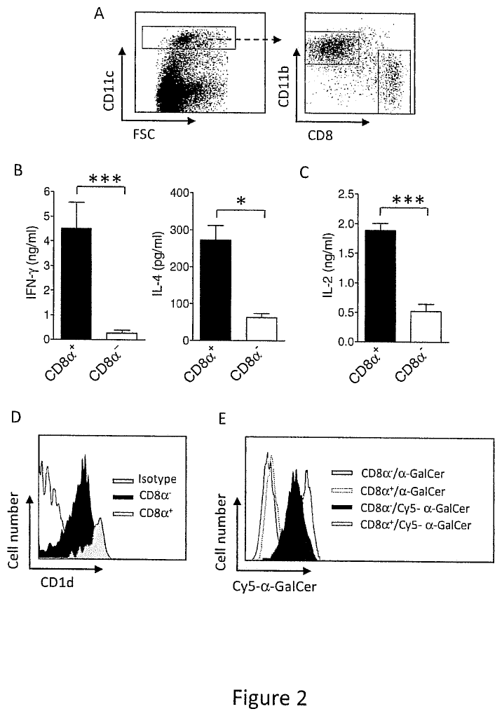

We then investigated the respective role of CD8α⁻ and CD8α⁺ DC subsets in the activation of iNKT cells. To address this issue, mice were inoculated with α-GalCer and 2 h later, splenic CD8α⁻ and CD8⁺ DCs were purified (FIG. 2A) and cultured with NKT cells. Relative to CD8α⁻ DCs, CD8α⁺ DCs promoted a much stronger secretion of IFN-7 and IL-4 (FIG. 2B). Similarly, CD8α⁺ DCs triggered a higher IL-2 production by the iNKT cell hybridoma DN32.D3, the activation of which depending solely on CD1d/Ag mediated TCR triggering (FIG. 2C). Flow cytometry analysis revealed a higher expression of CD1d on splenic CD8α⁺ DCs, compared to CD8α⁻ DCs (FIG. 2D). To investigate the possibility that the difference could also be due to a differential in vivo up-take of α-GalCer, Cy5-conjugated α-GalCer was administered. Relative to CD8α⁻ DCs, the incorporation rate of Cy5-conjugated α-GalCer was more important in CD8α⁺ DCs (FIG. 2E). On the contrary, exposure of spleen cells with Cy5-conjugated α-GalCer in vitro resulted in an identical uptake by both CD8α⁻ and CD8α⁺ DCs (data not shown). Collectively, upon systemic inoculation of free α-GalCer, CD8⁺ DCs are potent activators of iNKT cells.

The Delivery of α-GalCer into CD8α⁺ DCs Improves iNKT Cell Activation In Vitro and In Vivo

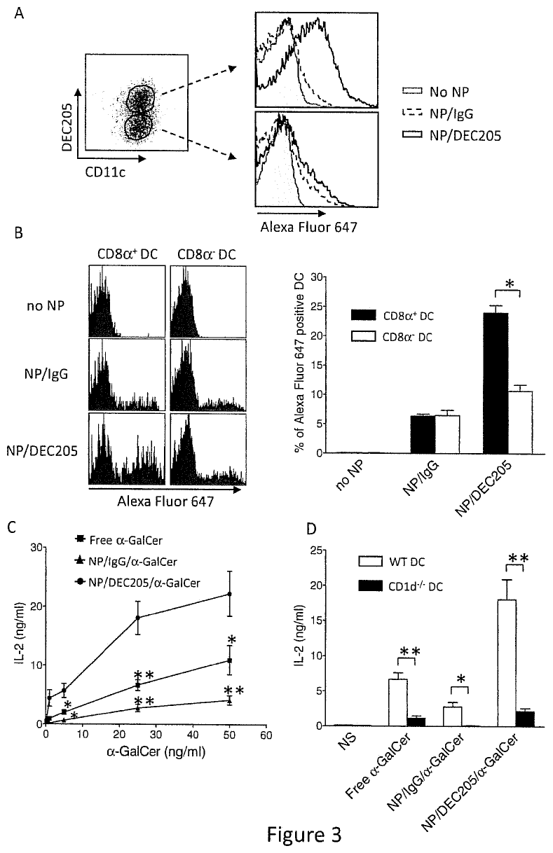

The endocytic C-type lectin receptor DEC205 is expressed on the cell surface of spleen and LN CD8α⁺ DCs.³⁶ We took advantage of this property to target α-GalCer into CD8α⁺ DCs. To do so, we formulated α-GalCer in PLGA-based NPs coated with Abs recognizing DEC205 (NP/DEC205) (for the physical and biochemical characteristics of NPs, see Table 1). As FIG. 3A shows, DEC205⁺ BM-DCs, in contrast to DEC205 BM-DCs, incorporated NP/DEC205 relative to NP/IgG, used here as a negative control (FIG. 3A). More importantly, when incubated with splenocytes, CD8α⁺ DCs more efficiently up-took NP/DEC205 relative to CD8α⁻ DCs (FIG. 3B). We then compared the biological activity of the formulations. BM-DCs incubated with grading doses of NP/DEC205/α-GalCer triggered a higher iNKT cell activation compared to free, non-vectorized, α-GalCer and particularly to NP/IgG/α-GalCer (FIG. 3C). As shown in FIG. 3D, the effect was CD1d dependent. These data collectively show that encapsulation of α-GalCer in NP/DEC205 selectively targets CD8α⁺ DCs to efficiently activate iNKT cells in vitro.

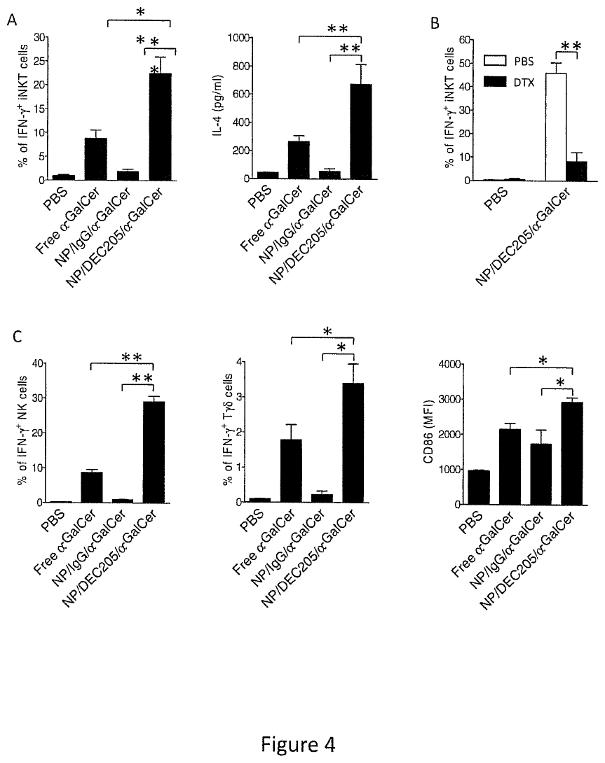

In vivo, NP/DEC205/α-GalCer promoted a higher iNKT cell activation relative to free α-GalCer, and particularly to NP/IgG/α-GalCer (5 ng α-GalCer/mouse) (FIG. 4A). Of note, DC depletion strongly reduced iNKT cell activation after NP/DEC205/α-GalCer administration (FIG. 4B). Primary activation of iNKT cells results in the trans-activation of other cell types, including NK cells, 78 T cells and DCs.^{7,37} As revealed in FIG. 4C, NP/DEC205/α-GalCer induced a higher level of IFN-γ production by NK cells and 75 T cells, relative to free α-GalCer. To a lesser extent, NP/DEC:205/α-GalCer triggered a higher level of CD86 expression by DCs. Thus, the in vivo delivery of α-GalCer into CD8α⁺ DCs is particularly potent to trigger iNKT cell-based transactivation of innate immune cells.

Targeting α-GalCer into CD8α⁺ DCs Prevents iNKT Cell Anergy In Vivo

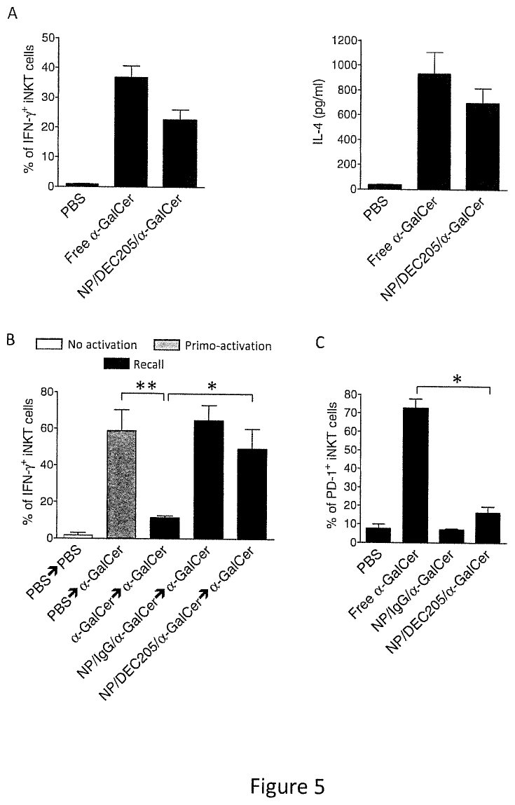

Having established that the in vivo delivery of α-GalCer into CD8α⁺ DCs enhanced the primary activation of iNKT cells, we next investigated whether it could impact on their responsiveness upon a recall response. To address this issue, mice were injected either with a low dose of α-GalCer encapsulated in NP/DEC205 (5 ng/mouse) or a high dose of free α-GalCer (100 ng/mouse), both leading to a comparable primary iNKT cell activation (FIG. 5A). Strikingly, whilst free α-GalCer induced iNKT cell anergy, NP/DEC205/α-GalCer failed to do so (FIG. 5B). It is noticeable that, in this condition, the frequency of IFN-γ positive iNKT cells was comparable to that in animals injected once with a high dose of α-GalCer. Finally, whereas free α-GalCer induced PD-1 expression on iNKT cells, this was not the case after NP/DEC205/α-GalCer administration (FIG. 5C). Collectively, in vivo delivery of α-GalCer into CD8α⁺ DCs prevents iNKT cell anergy.

The co-delivery of α-GalCer and OVA into CD8α⁺ DCs optimizes CD8α⁺ T Cell and Ab Responses

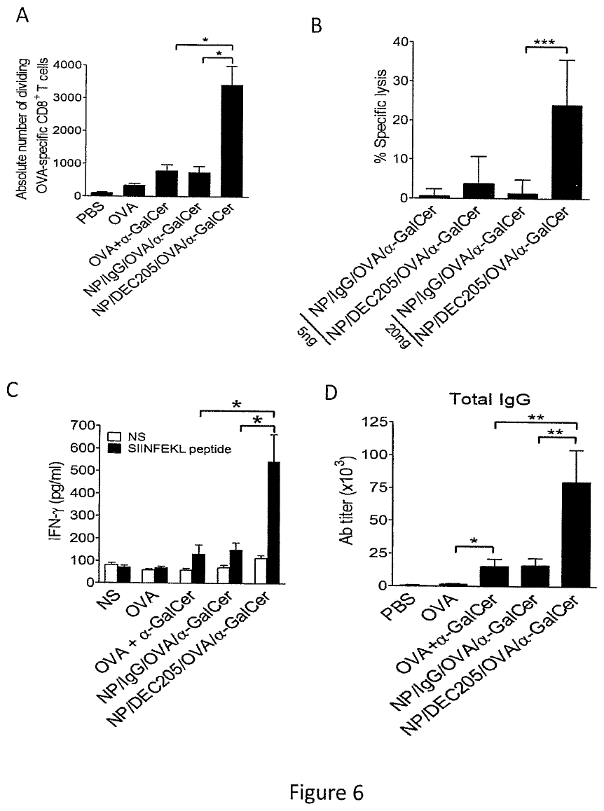

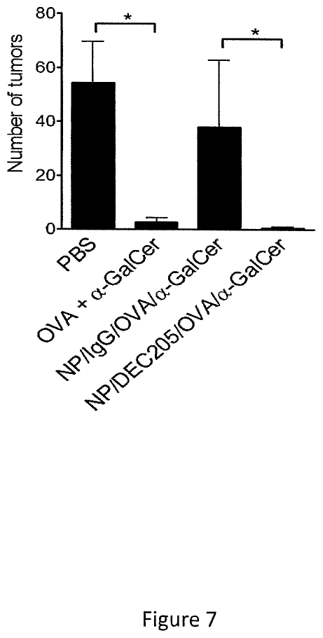

Numerous studies in mice have shown a benefit to target Ags to CD8α⁺ DCs via DEC205, particularly to promote Ag cross-presentation and to prime CD8 T cells.^{38,39} The effects of targeting α-GalCer and Ag in intimate association with each other to CD8α⁻ DCs have not yet been investigated. To do so, mice reconstituted with CFSE-labelled OT-I cells were inoculated with NP/DEC205 containing both α-GalCer and the model Ag ovalbumin (OVA). NP/DEC205/OVA/α-GalCer induced a higher proliferation of OVA-specific OT-I cells compared to mice inoculated with NP/IgG/OVA/α-GalCer or with soluble α-GalCer plus OVA or OVA alone (FIG. 6A and FIG. 7). Furthermore, upon in vitro peptide restimulation, the frequency of OVA-specific CD8⁺ T lymphocytes expressing IFN-γ was higher in mice that received NP/DEC205/OVA/α-GalCer, compared to other animal groups (Fig S1B). NP/DEC205/OVA/α-GalCer also elicited a higher cytotoxic T cell activity, as assessed by the measurement of target cell lysis (FIG. 6B). Finally, whilst the recall response two months after the last immunization was nearly undetectable in other groups, mice administered with NP/DEC205/OVA/α-GalCer displayed a long-lasting CD8⁺ T cell memory response (FIG. 6C). Targeting DEC205 can also promote humoral responses.

^{.40,41} Indeed, relative to OVA plus α-GalCer, NP/DEC205/OVA/α-GalCer promoted a higher tiler of OVA-specific IgG (FIG. 6D). Thus, combining OVA and α-GalCer into the same particle to target CD8α⁺ DCs via DEC205 is clearly of benefit to enhance cellular and humoral immune responses.

NPs Incorporating α-GalCer and Ag Protect Against Tumor Development.

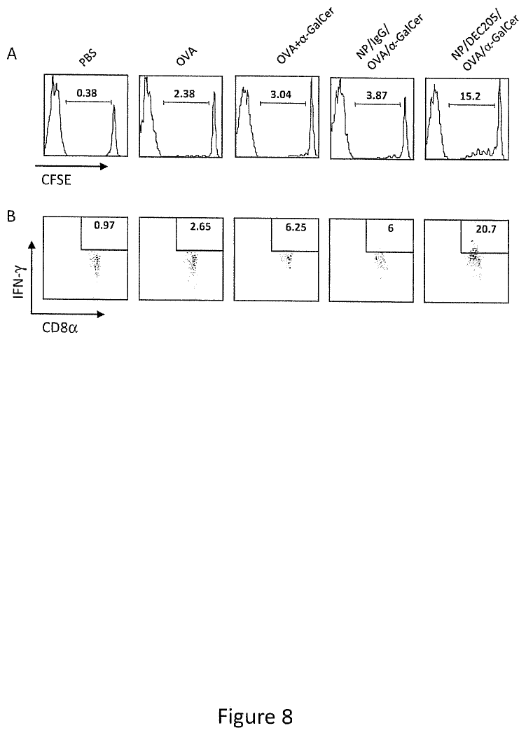

To investigate the consequences of α-GalCer and Ag vectorization on the control of tumor development, mice were vaccinated with NP/DEC205/OVA/α-GalCer before inoculation of OVA-expressing B16F10 melanoma cells. As a positive control, mice received a high dose of free α-GalCer plus OVA (10-fold more). As FIG. 8 shows, and compared to mice receiving NP/IgG/OVA/α-GalCer, vaccinated mice were fully protected against the development of lung metastases.

Discussion

Alpha-GalCer is a strong immunostimulatory molecule holding great promises for therapeutic purposes and vaccine development.^{1,2,7,42} Several concerns however limit its use in clinics. Among them, the still unknown nature of cells α-GalCer targets, and thus the uncontrolled response it promotes, remains a major issue. Most importantly is the profound and long term iNKT cell unresponsiveness α-GalCer induces, a major hurdle for patients needing several immunological stimuli to develop effective (e.g. anti-tumoral) responses.^{22,24,26,43} A possibility to better control iNKT cell functions might lie on passive or active delivery of α-GalCer into the right APCs, such as DCs. This strategy might also enhance the strength and the quality of iNKT cell-mediated immune responses. The inventors and others have shown that α-GalCer vectorized in PLGA-based NPs³² or liposomes (data not shown) or included into virus-like particles⁴⁴ activated iNKT cells but failed to prevent their anergy upon re-stimulation. Therefore, a controlled delivery of α-GalCer is a requisite to optimize iNKT cell responses. In the present invention, the inventors demonstrate for the first time that specific delivery of α-GalCer into CD8α⁺ DCs is instrumental to enhance primary activation of iNKT cells and to avoid iNKT cell anergy. In parallel, co-delivery of α-GalCer and Ag into CD8α⁺ DCs (likely to be the same DCs) critically exacerbates cellular and humoral immune responses.

The data indicated that DCs are primary initiators of iNKT cell activation after systemic administration of non-vectorized α-GalCer, in line with other reports.⁸⁻¹² Dendritic cells from the spleen are heterogeneous and recent studies suggested that DC subsets could differ in their ability to stimulate iNKT cells.^{18,20,29} For example, it has been previously reported that amongst the CD8α⁻ DC subset, CD4⁻ DCs were more efficient at activating iNKT cells, relative to CD4⁺ DCs.²⁹ Consistent with the higher α-GalCer uptake rate in vivo and the enhanced level of cell surface CD1d, the results show that, relative to CD8α⁻ DCs, CD8α⁺ DCs are potent triggers of iNKT cell activation. The fact that a large number of CD80α⁺ DCs and iNKT cells co-localize in the marginal zone of the spleen12,36,45,46 is in line with such observation. The impact of α-GalCer delivery into CD8⁺ DCs on immune responses was then investigated. NP/DEC205 specifically target splenic CD8α⁺ (DEC205⁺) DCs and α-GalCer vectorized in NP/DEC205 can be loaded onto CD1d and presented to and activate iNKT cells. Of importance, NP/DEC205/α-GalCer was much more efficient at activating iNKT cells in vitro and in vivo, relative to free α-GalCer and to NP/IgG/α-GalCer, a process that depended on DCs (FIG. 4B). The enhanced iNKT cell response is probably due to the rapid uptake of NPs, and thus α-GalCer, by CD8α⁺ DCs. It is also likely that the DEC205-mediated incorporation of NPs facilitates α-GalCer accessibility to the CD1d molecule in endosomes/lysosomes of DCs. Of interest, delivery of α-GalCer into CD8α⁺ DCs also increased the trans-activation of NK, γδ T lymphocytes and DCs. Thus, CD8α⁺ DCs targeting through DEC205 optimizes the iNKT cell-mediated innate immune response.

Following primary activation, iNKT cells develop a long-lasting hyporesponsiveness thereby preventing activation upon repeated exposure to α-GalCer.¹¹ The potential role played by DCs in iNKT cell anergy is still debated.^{10,11,23} Using two complementary strategies (CD11c-DTR mice and DC transfer), the results clearly show that presentation of α-GalCer by DCs (primary iNKT cell activation) does not lead to iNKT cell anergy after secondary stimulation, in line with other studies.^{10,11} Paralleling this, targeting α-GalCer to CD8α⁺ DCs by means of NP/DEC205 did not promote iNKT cell unresponsiveness, confirming the key role played by DCs in maintaining secondary iNKT cell activation. Thus, α-GalCer-loaded CD8α⁺ DCs not only efficiently trigger TCR signalling in iNKT cells (primo-stimulation) but also maintain secondary activation after challenge. During primary iNKT cell activation, multiple signals from surface-bound and soluble costimulatory and/or inhibitory molecules function in concert to stimulate and fine-tune the iNKT cell response. It is thus likely that, in addition to CD1d, CD8α⁺ DCs provide additional signals, absent in CD11c non-expressing cells (e.g. B lymphocytes), to maintain secondary iNKT cell activation.

Co-delivering Ag and adjuvants into DCs, including CD8α⁺ DCs, has been shown to induce optimal T and B cell-mediated immune responses.⁴⁷⁻⁴⁹ Whether the co-delivery of α-GalCer and Ag in a specific subset of DCs impacts on the immune response has not yet been studied. TLR agonists exert adjuvant effects by inducing direct DC maturation, a process that lowers Ag up-take but is crucial to efficiently prime nave T cells. In the case of α-GalCer, DC maturation is indirect and lies on iNKT cell factors produced following primo-activation. It is possible that the delayed maturation of DCs in response α-GalCer, relative to TLR agonists, might prolong the Ag uptake capacity of DCs, thus leading to amplified immune responses. The inventors have shown that co-delivery of α-GalCer and OVA into CD8α⁺ DCs amplified the early and late CD8⁺ T cell responses compared to the same amount of α-GalCer and OVA given in untargeted form. CD8⁺ DCs excel in MHC class I cross-presentation and iNKT cells have been shown to directly licence CD8α⁺ DCs for cross priming, even in the absence of CD4⁺ T cells.⁵⁰ To the inventor's knowledge, the current report is the first to experimentally prove the benefit of α-GalCer and Ag co-delivery into CD8α⁺ DCs in order to enhance the CDR T cell responses. In the same vein, the OVA-specific Ab response was greatly augmented in response to α-GalCer and Ag inserted into the same particle and targeted to CD8α⁺ DCs. Thus, as shown for TLR agonists,⁴⁹ targeting α-GalCer to CD8α⁺ DCs improves α-GalCer adjuvanticity. Although this remains to be fully demonstrated, we hypothesize that α-GalCer/Ag co-delivery to CD8α⁺ DCs might have superior effects compared to TLR agonist/Ag co-delivery. Indeed, TLR agonists exert adjuvant effects by inducing direct DC maturation, a process that lowers Ag up-take but is crucial to efficiently prime nave T cells. In the case of α-GalCer, DC maturation is indirect and lies on iNKT cell factors produced following primo-activation. It is possible that the delayed maturation of DCs in response to α-GalCer, relative to TLR agonists, might prolong the Ag uptake capacity of DCs, thus leading to amplified immune responses. The fact that iNKT cells can substitute CD4⁺ T helper cells to induce T and B cell responses offers a new avenue for investigating the consequences of iNKT cell-based adjuvant properties in many settings. Our data further reveals that α-GalCer/Ag co-delivery to CD8α+ DCs triggers a potent anti-tumor response.

To conclude, we have designed and undertaken an active targeting strategy based on the use of PLGA-based NPs carrying on their surface DC-specific Abs in order to minimize the unwanted effects of uncontrolled iNKT cell activation, while maximizing the ability of iNKT cells to promote efficient adaptive and anti-tumor immune responses. We show for the first time that the in vivo delivery of α-GalCer into CD8α+ DCs enhances the early activation of iNKT cells but also allows these cells to respond to further re-stimulations. Of importance, the NP-mediated simultaneous co-delivery of both α-GalCer and protein Ag in CD8α+ DCs induces optimal CD8+ T cell and anti-tumor responses in the mouse system.

### REFERENCES

1. Bendelac A, Savage P B, Teyton L. The biology of NKT cells. Annu Rev Immunol. 2007; 25:297-336.

2. Cerundolo V, Silk J D, Masri S H, Salio M. Harnessing invariant NKT cells in vaccination strategies. Nat Rev Immunol. 2009; 9:28-38.

3. Van Kaer L, Joyce S. Innate immunity: NKT cells in the spotlight. Curr Biol. 2005; 15:R429-431.

4. Girardi E, Zajonc D M. Molecular basis of lipid antigen presentation by CD1d and recognition by natural killer T cells. Immunol Rev. 2012; 250:167-179.

5. Rossjohn J, Pellicci D G, Patel O, Gapin L, Godfrey D I. Recognition of CD1d-restricted antigens by natural killer T cells. Nat Rev Immunol. 2012; 12:845-857.

6. Paget C, Chow M T, Duret H, Mattarollo S R, Smyth M J. Role of gammadelta T cells in alpha-galactosylceramide-mediated immunity. J Immunol. 2012; 188:3928-3939.

7. Fujii S, Motohashi S, Shimizu K, Nakayama T, Yoshiga Y. Taniguchi M. Adjuvant activity mediated by iNKT cells. Semin Immunol. 2010; 22:97-102.

8. Bai L, Constantinides M G, Thomas S Y, et al. Distinct APCs explain the cytokine bias of alpha-galactosylceramide variants in vivo. J Immunol. 2012; 1 88: 3053-3061.

9. Bezbradica J S, Stanic A K, Matsuki N, et al. Distinct roles of dendritic cells and B cells in Va14Ja18 natural T cell activation in vivo. J Immunol. 2005: 174:4696-4705.

10. Fujii S, Shimizu K, Kronenberg M, Steinman R M. Prolonged IFN-gamma-producing NKT response induced with alpha-galactosylceramide-loaded DCs. Nat Immunol. 2002; 3:867-874.

11. Parekh V V, Wilson M T, Olivares-Villagomez D, et al. Glycolipid antigen induces long-term natural killer T cell anergy in mice. J Clin Invest. 2005; 115:2572-2583.

12. Barral P, Sanchez-Nino M D, van Rooijen N, Cerundolo V, Batista F D. The location of splenic NKT cells favours their rapid activation by blood-borne antigen. Embo J. 2012; 31:3029-3030.

13. Dudziak D, Kamphorst A O, Heidkamp G F, et al. Differential antigen processing by dendritic cell subsets in vivo. Science. 2007; 315:107-111.

14. Shortman K, Liu Y J. Mouse and human dendritic cell subtypes. Nat Rev Immunol. 2002: 2:151-161.

15. Mebius R E, Kraal G. Structure and function of the spleen. Nat Rev Immunol. 2005: 5:606-616.

16. Shortman K, Heath W R. The CD8+ dendritic cell subset. Immunol Rev. 2010: 234:18-31.

17. Vremec D, Pooley Hochrein H. Wu L. Shortman K. CD4 and CD8 expression by dendritic cell subtypes in mouse thymus and spleen. J Immunol. 2000; 164:2978-2986.

18. Farrand K J, Dickgreber N, Stoitzner P, Ronchese F, Petersen T R, Hermans I F. Langerin+CD8alpha+dendritic cells are critical for cross-priming and IL-12 production in response to systemic antigens. J Immunol. 2009; 183:7732-7742.

19. Villadangos J A, Schnorrer P. Intrinsic and cooperative antigen-presenting functions of dendritic-cell subsets in vivo. Nat Rev Immunol. 2007: 7:543-555.

20. Simkins H M, Hyde E, Farrand K J, et al. Administration of alpha-galactosylceramide impairs the survival of dendritic cell subpopulations in vivo. J Leukoc Biol. 2011.

21. Giaccone G, Punt C J, Ando Y. et al. A phase I study of the natural killer T-cell ligand alpha-galactosylceramide (KRN7000) in patients with solid tumors. Clin Cancer Res. 2002; 8:3702-3709.

22. Schneiders F L, Scheper R J, von Blomberg B M, et al. Clinical experience with α-galactosylceramide (KRN7000) in patients with advanced cancer and chronic hepatitis B/C infection. Clin Immunol. 2011; 140:130-141.

23. Iyoda t, Ushida M, Kimura Y, et al. Invariant NKT cell anergy is induced by a strong TCR-mediated signal plus co-stimulation. Int Immunol. 2010; 22:905-913.

24. Dhodapkar M V, Richter J. Harnessing natural killer T (NKT) cells in human myeloma: progress and challenges. Clin Immunol. 2011; 140:160-166.

25. Motohashi S, Nakayama T. Invariant natural killer T cell-based immunotherapy for cancer. Immunotherapy. 2009; 1:73-82.

26. Yamasaki K, Horiguchi S, Kurosaki M, et al. Induction of NKT cell-specific immune responses in cancer tissues after NKT cell-targeted adoptive immunotherapy. Clin Immunol. 2011; 138:255-265.

27. Jung S, Unutmaz D, Wong P, et al. In vivo depletion of CD11c+dendritic cells abrogates priming of CD8+ T cells by exogenous cell-associated antigens. Immunity. 2002; 17:211-220.

28. Mendiratta S K, Martin W D, Hong S, Boesteanu A, Joyce S, Van Kaer L. CD1d1 mutant mice are deficient in natural T cells that promptly produce IL-4. Immunity. 1997; 6:469-477.

29. Bialecki E, Macho Fernandez E, Ivanov S, et al. Spleen-resident CD4+ and CD4-CD8alpha-dendritic cell subsets differ in their ability to prime invariant natural killer T lymphocytes. PLoS One. 2011: 6:e26919.

30. Cruz L J, Tacken P J, Bonetto F. et al. Multimodal imaging of nanovaccine carriers targeted to human dendritic cells. Mol Pharm. 2011; 8:520-531.

31. Cruz U. Tacken P J, Fokkink R, et al. Targeted PLGA nano—but not microparticles specifically deliver antigen to human dendritic cells via DC-SIGN in vitro. J Control Release. 2010; 144:118-126.

32. Macho Fernandez E, Chang J, Fontaine J, et al. Activation of invariant Natural Killer T lymphocytes in response to the alpha-galactosylceramide analogue KRN7000 encapsulated in PLGA-based nanoparticles and microparticles. Int. J. Pharm. 2012; 423:45-54.

33. Chang W S. Kim J Y, Kim Y J, et al. Cutting edge: Programmed death-I/programmed death ligand 1 interaction regulates the induction and maintenance of invariant NKT cell anergy. J Immunol. 2008; 181:6707-6710.

34. Courtney A N, Thapa P, Singh S, Wishahy A M, Zhou D, Sastry J. Intranasal hut not intravenous delivery of the adjuvant alpha-galactosylceramide permits repeated stimulation of natural killer T cells in the lung. Eur J. Immunol. 2011; 41:3312-3322.

35. Parekh V V, Lalani S, Kim S, et al. PD-1/PD-L blockade prevents anergy induction and enhances the anti-tumor activities of glycolipid-activated invariant NKT cells. J Immunol. 2009; 182:2816-2826.

36. Idoyaga L Suda N, Suda K. Park C G, Steinman R M. Antibody to Langerin/CD207 localizes large numbers of CD8alpha+ dendritic cells to the marginal zone of mouse spleen. Proc Natl Acad Sci U S A. 2009; 106:1524-1529.

37. Paget C, Mallevaey T, Speak A O. et al. Activation of invariant NKT cells by toll-like receptor 9-stimulated dendritic cells requires type I interferon and charged glycosphingolipids. Immunity. 2007; 27:597-609.

38. Bonifaz L C, Bonnyay D P, Charalambous A. et al. In vivo targeting of antigens to maturing dendritic cells via the DEC-205 receptor improves T cell vaccination. J Exp Med. 2004; 199:815-824.

39. Idoyaga J, Lubkin A, Fiorese C, et al. Comparable T helper 1 (Th1) and CD8 T-cell immunity by targeting HIV gag p24 to CD8 dendritic cells within antibodies to Langerin, DEC205, and Clec9A. Proc Natl Acad Sci U S A. 2011; 108:2384-2389.

40. Birkholz K, Schwenkert M, Kellner C, et al. Targeting of DEC-205 on human dendritic cells results in efficient MIIC class II-restricted antigen presentation. Blood. 2010; 116:2277-2285.

41. Cheong C, Choi J H, Vitale L, et al. Improved cellular and humoral immune responses in vivo following targeting of HIV Gag to dendritic cells within human anti-human DEC205 monoclonal antibody. Blood. 2010; 116:3828-3838.

42. Berzins S P, Smyth M J. Baxter A G. Presumed guilty: natural killer T cell defects and human disease. Nat Rev Immunol. 2011; 11:131-142.

43. Motohashi S, Okamoto Y, Yoshino I, Nakayama T. Anti-tumor immune responses induced by iNKT cell-based immunotherapy for lung cancer and head and neck cancer. Clin Immunol. 2011; 140:167-176.

44. McKee S J, Young V L, Clow F, et al. Virus-like particles and alpha-galactosylceramide form a self-adjuvanting composite particle that elicits anti-tumor responses. J Control Release. 2012; 159:338-345.

45. Edelson B T, Bradstreet T R, Hildner K, et al. CD8alpha(+) dendritic cells are an obligate cellular entry point for productive infection by Listeria monocytogenes. Immunity. 2011; 35:236-248.

46. Qiu C H, Miyake Y, Kaise H, Kitamura H, Ohara O, Tanaka M. Novel subset of CD8α⁺ dendritic cells localized in the marginal zone is responsible for tolerance to cell-associated antigens. J Immunol. 2009; 182:4127-4136.

47. Bal S M, Hortensius S, Ding Z. Jiskoot W, Bouwstra J A. Co-encapsulation of antigen and Toll-like receptor ligand in cationic liposomes affects the quality of the immune response in mice after intradermal vaccination. Vaccine. 2011; 29:1045-1052.

48. Schlosser E, Mueller M, Fischer S, et al. TLR ligands and antigen need to be coencapsulated into the same biodegradable microsphere for the generation of potent cytotoxic T lymphocyte responses. Vaccine. 2008; 26:1626-1637.

49. Tacken P J, Zeelenberg I S, Cruz L J, et al. Targeted delivery of TLR ligands to human and mouse dendritic cells strongly enhances adjuvanticity. Blood. 2011; 118:6836-6844.

50. Semmling V. Lukacs-Kornek V, Thaiss C A, et al. Alternative cross-priming through CCL17-CCR4-mediated attraction of CTLs toward NKT cell-licensed DCs. Nat Immunol. 2010: 11:313-320.

51. Macho Fernandez, E., J. Chang, J. Fontaine, E. Bialecki, F. Rodriguez, E. Werkmeister, V. Krieger, C. Ehret, B. Heurtault, S. Fournel, B. Frisch, D. Betheder, C. Faveeuw, and F. Trottein. 2012. Activation of invariant Natural Killer T lymphocytes in response to the alpha-galactosylceramide analogue KRN7000 encapsulated in PLGA-based nanoparticles and microparticles. Int. J. Pharm. 423: 45-54.

1. A method for treating a tumor in a subject in need thereof, said method comprising administering a therapeutically efficient amount of a nanoparticle comprising:
i. a core containing polymers,
ii. a coating,
iii. an α-GalCer compound,
iv. one or more antigenic determinant(s) specific for said tumor cell, and
v. a targeting agent comprising an antibody or its antigen-binding fragment that specifically binds to a cell surface marker specific of human BDCA-3+ dendritic cells,
wherein said nanoparticle does not comprise a CD1d molecule, and wherein said targeting agent is covalently linked to a surface of the coating.

2. The method of claim 1, wherein said nanoparticle has a size between 10 to 2000 nm diameter.

3. The method of claim 1, wherein said core comprises poly(lactic acid), poly(glycolic acid), or their co-polymers.

4. The method of claim 1, wherein said α-GalCer compound is α-galactosylceramide or its functional derivatives.

5. The method of claim 1, wherein said α-GalCer compound is α-galactosylceramide or its functional derivatives.

6. The method of claim 1, wherein said α-GalCer compound and said one or more antigenic determinant(s) are encapsulated within the nanoparticle.

7. The method of claim 1, wherein said α-GalCer compound and said one or more antigenic determinant(s) are physically coupled to the nanoparticles.

8. The method of claim 1, wherein said cell surface marker specific human BDCA3+ is CLEC9A.


SEQUENCE LISTING (3 sequences)

SEQ ID NO: 1 - protein, 241 aa, Homo sapiens
MHEEEIYTSLQWDSPAPDTYQKCLSSNKCSGACCLVMVISCVFCMGLLTASIFLGVKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 2 - protein, 333 aa, Homo sapiens
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 3 - protein, 8 aa, Artificial Sequence
  Synthetic OVA peptide
SIINFEKL